SEQUENCE LISTING (4 sequences)

SEQ ID NO: 1 - protein, 470 aa, Homo sapiens
MAALGVQSINWQTAFNRQAHHTDKFSSQELILRRGQNFQVLMIMNKGLGSNERLEFIVSTGPYPSESAMTKAVFPLSNGSSGGWSAVLQASNGNTLTISISSPASAPIGRYTMALQIFSQGGISSVKLGTFILLFNPWLNVDSVFMGNHAEREEYVQEDAGIIFVGSTNRIGMIGWNFGQFEEDILSICLSILDRSLNFRRDAATDVASRNDPKYVGRVLSAMINSNDDNGVLAGNWSGTYTGGRDPRSWNGSVEILKNWKKSGFSPVRYGQCWVFAGTLNTALRSLGIPSRVITNFNSAHDTDRNLSVDVYYDPMGNPLDKGSDSVWNFHVWNEGWFVRSDLGPSYGGWQVLDATPQERSQGVFQCGPASVIGVREGDVQLNFDMPFIFAEVNADRITWLYDNTTGKQWKNSVNSHTIGRYISTKAVGSNARMDVTDKYKYPEGSDQERQVFQKALGKLKPNTPFAATS

SEQ ID NO: 2 - DNA, 1411 bp, Homo sapiens
atggctgctctaggagtccagagtatcaactggcagacggccttcaaccgacaagcgcatcacacagacaagttctccagccaggagctcatcttgcggagaggccaaaacttccaggtcttaatgatcatgaacaaaggccttggctctaacgaaagactggagttcattgtctccacagggccttacccctcagagtcggccatgacgaaggctgtgtttccactctccaatggcagtagtggtggctggagtgcggtgcttcaggccagcaatggcaatactctgactatcagcatctccagtcctgccagcgcacccataggacggtacacaatggccctccagatcttctcccagggcggcatctcctctgtgaaacttgggacgttcatactgctttttaacccctggctgaatgtggatagcgtctttatgggtaaccacgctgagagagaagagtatgttcaggaagatgccggcatcatctttgtgggaagcacaaaccgaattggcatgattggctggaactttggacagtttgaagaagacattctcagcatctgcctctcaatcttggataggagtctgaatttccgccgtgacgctgctactgatgtggccagcagaaatgaccccaaatacgttggccgggtgctgagtgccatgatcaatagcaatgatgacaatggtgtgcttgctgggaattggagcggcacttacaccggtggccgggacccaaggagctggaacggcagcgtggagatcctcaaaaattggaaaaaatctggcttcagcccagtccgatatggccagtgctgggtctttgctgggaccctcaacacagcgctgcggtctttggggattccttcccgggtgatcaccaacttcaactcagctcatgacacagaccgaaatctcagtgtggatgtgtactacgaccccatgggaaaccccctggacaagggtagtgatagcgtatggaatttccatgtctggaatgaaggctggtttgtgaggtctgacctgggcccctcgtacggtggatggcaggtgttggatgctaccccgcaggaaagaagccaaggggtgttccagtgcggccccgcttcggtcattggtgttcgagagggtgatgtgcagctgaacttcgacatgccctttatcttcgcggaggttaatgccgaccgcatcacctggctgtacgacaacaccactggcaaacagtggaagaattccgtgaacagtcacaccattggcaggtacatcagcaccaaggcggtgggcagcaatgctcgcatggacgtcacggacaagtacaagtacccagaaggctctgaccaggaaagacaagtgttccaaaaggctttggggaaacttaaacccaacacgccatttgccgcgacgtctt

SEQ ID NO: 3 - protein, 693 aa, Homo sapiens
MAALGVQSINWQTAFNRQAHHTDKFSSQELILRRGQNFQVLMIMNKGLGSNERLEFIVSTGPYPSESAMTKAVFPLSNGSSGGWSAVLQASNGNTLTISISSPASAPIGRYTMALQIFSQGGISSVKLGTFILLFNPWLNVDSVFMGNHAEREEYVQEDAGIIFVGSTNRIGMIGWNFGQFEEDILSICLSILDRSLNFRRDAATDVASRNDPKYVGRVLSAMINSNDDNGVLAGNWSGTYTGGRDPRSWNGSVEILKNWKKSGFSPVRYGQCWVFAGTLNTALRSLGIPSRVITNFNSAHDTDRNLSVDVYYDPMGNPLDKGSDSVWNFHVWNEGWFVRSDLGPSYGGWQVLDATPQERSQGVFQCGPASVIGVREGDVQLNFDMPFIFAEVNADRITWLYDNTTGKQWKNSVNSHTIGRYISTKAVGSNARMDVTDKYKYPEGSDQERQVFQKALGKLKPNTPFAATSSMGLETEEQEPSIIGKLKVAGMLAVGKEVNLVLLLKNLSRDTKTVTVNMTAWTIIYNGTLVHEVWKDSATMSLDPEEEAEHPIKISYAQYEKYLKSDNMIRITAVCKVPDESEVVVERDIILDNPTLTLEVLNEARVRKPVNVQMLFSNPLDEPVRDCVLMVEGSGLLLGNLKIDVPTLGPKEGSRVRFDILPSRSGTKQLLADFSCNKFPAIKAMLSIDVAE

SEQ ID NO: 4 - DNA, 2082 bp, Homo sapiens
atggctgctctaggagtccagagtatcaactggcagacggccttcaaccgacaagcgcatcacacagacaagttctccagccaggagctcatcttgcggagaggccaaaacttccaggtcttaatgatcatgaacaaaggccttggctctaacgaaagactggagttcattgtctccacagggccttacccctcagagtcggccatgacgaaggctgtgtttccactctccaatggcagtagtggtggctggagtgcggtgcttcaggccagcaatggcaatactctgactatcagcatctccagtcctgccagcgcacccataggacggtacacaatggccctccagatcttctcccagggcggcatctcctctgtgaaacttgggacgttcatactgctttttaacccctggctgaatgtggatagcgtctttatgggtaaccacgctgagagagaagagtatgttcaggaagatgccggcatcatctttgtgggaagcacaaaccgaattggcatgattggctggaactttggacagtttgaagaagacattctcagcatctgcctctcaatcttggataggagtctgaatttccgccgtgacgctgctactgatgtggccagcagaaatgaccccaaatacgttggccgggtgctgagtgccatgatcaatagcaatgatgacaatggtgtgcttgctgggaattggagcggcacttacaccggtggccgggacccaaggagctggaacggcagcgtggagatcctcaaaaattggaaaaaatctggcttcagcccagtccgatatggccagtgctgggtctttgctgggaccctcaacacagcgctgcggtctttggggattccttcccgggtgatcaccaacttcaactcagctcatgacacagaccgaaatctcagtgtggatgtgtactacgaccccatgggaaaccccctggacaagggtagtgatagcgtatggaatttccatgtctggaatgaaggctggtttgtgaggtctgacctgggcccctcgtacggtggatggcaggtgttggatgctaccccgcaggaaagaagccaaggggtgttccagtgcggccccgcttcggtcattggtgttcgagagggtgatgtgcagctgaacttcgacatgccctttatcttcgcggaggttaatgccgaccgcatcacctggctgtacgacaacaccactggcaaacagtggaagaattccgtgaacagtcacaccattggcaggtacatcagcaccaaggcggtgggcagcaatgctcgcatggacgtcacggacaagtacaagtacccagaaggctctgaccaggaaagacaagtgttccaaaaggctttggggaaacttaaacccaacacgccatttgccgcgacgtcttcaatgggtttggaaacagaggaacaggagcccagcatcatcgggaagctgaaggtcgctggcatgctggcagtaggcaaagaagtcaacctggtcctactgctcaaaaacctgagcagggatacgaagacagtgacagtgaacatgacagcctggaccatcatctacaacggcacgcttgtacatgaagtgtggaaggactctgccacaatgtccctggaccctgaggaagaggcagaacatcccataaagatctcgtacgctcagtatgagaagtacctgaagtcagacaacatgatccggatcacagcggtgtgcaaggtcccagatgagtctgaggtggtggtggagcgggacatcatcctggacaaccccaccttgaccctggaggtgctgaacgaggctcgtgtgcggaagcctgtgaacgtgcagatgctcttctccaatccactggatgagccggtgagggactgcgtgctgatggtggagggaagcggcctgctgttgggtaacctgaagatcgacgtgccgaccctagggcccaaggaggggtcccgggtccgttttgatatcctgccctcccggagtggcaccaagcaactgctcgccgacttctcctgcaacaagttccctgcaatcaaggccatgttgtccatcgatgtagccgaatga

(19) United States
(12) Patent Application Publication
(10) Pub. No.: US 2024/0044897 A1
(43) Pub. Date: Feb. 8, 2024

(54) METHOD OF PROGNOSIS AND/OR DIAGNOSIS OF A PELLICULAR CONDITION OF THE SCALP

(71) Applicant: L’OREAL, Paris (FR)

(72) Inventors: Namita MISRA, AULNAY-SOUS-BOIS (FR); Nükhet CAVUSOGLU, AULNAY-SOUS-BOIS (FR); Aude FOUCHER, AULNAY-SOUS-BOIS (FR)

(21) Appl. No.: 18/266,296

(22) PCT Filed: Dec. 15, 2021
(86) PCT No.: PCT/EP2021/086015

§ 371 Date: Jun. 9, 2023

(30) Foreign Application Priority Data
Dec. 16, 2020 (FR) 2013349

(51) Int. Cl. G01N 33/573

(52) U.S. Cl. CPC G01N 33/573; G01N 2333/91085; G01N 2800/20; G01N 2800/50; G01N 2500/04

(57) ABSTRACT

The present invention relates to a method of prognosis and/or diagnosis of a pellicular condition of the scalp, based on the measurement of the quantity of the active form of transglutaminase 3. The present invention also relates to a cosmetic treatment method for the scalp, a method for identifying compounds for reducing and/or eliminating the pellicular condition. Finally, the present invention relates to a kit and the use thereof for identifying a compound for reducing and/or eliminating the pellicular condition.

The present invention relates to a method of prognosis and/or diagnosis of a pellicular condition of the scalp, based on the measurement of the quantity of the active form of transglutaminase 3. The present invention also relates to a cosmetic treatment method for the scalp, a method for identifying compounds for reducing and/or eliminating the pellicular condition. Finally, the present invention relates to a kit and the use thereof for identifying a compound for reducing and/or eliminating the pellicular condition.

The skin, and particularly the scalp, is a continuously renewed epithelium. Renewal, or desquamation, is a coordinated and finely regulated process leading to the elimination of surface cells, insensibly and invisibly. However, for various reasons, abnormal or irregular desquamation of the cells in the stratum corneum can lead to the formation of large, thick clumps of cells, visible to the naked eye and called “dander”, or “dandruff” in the context of the scalp, or in other situations, to thinning of the stratum corneum.

By way of example of factors promoting the appearance of dander or dandruff, mention can be made of stress, the winter season, excess sebum, lack of hydration, and/or colonization of the skin or hair follicles by the yeast Malassezia sp. In particular, Malassezia sp. type yeasts, forming part of the normal commensal flora on the surface of the scalp in dandruff-free subjects, see the proportion thereof increase substantially in cases of dandruff.

The presence of dander or pellicular conditions can be chronic, frequent, recurrent, and socially incapacitating conditions due to the obvious unsightly nature thereof. Moreover, pellicular conditions of the scalp or abnormal desquamation of the skin can result in itching sensations, resulting in scratching behaviors amplifying the phenomenon of the appearance of dander or dandruff.

Pellicular conditions of the scalp can be of greasy type or of dry type. Dry pellicular conditions of the scalp occur more frequently. The scalp being rich in sebaceous glands, a pellicular condition can develop more easily in the excessive presence of sebum. Thus, excessive sebum secretion, or hyperseborrhea, promotes the appearance of a greasy pellicular condition of the scalp, or greasy dandruff, generally associated with annoyances, sensations of discomfort, and esthetic disorders. Pellicular conditions respond, as a general rule, to various local or systemic treatments. However, the effectiveness of these treatments is merely suspensory and often involves thorough follow-up by the user (sufficient frequency of use and application time). However, daily and long-term use of these treatments can give rise to a habituation phenomenon reducing the effectiveness thereof, and generally associated with a rebound phenomenon occurring on treatment cessation. This phenomenon manifests as hyperseborrhea, worsening the pellicular condition. Moreover, the aggressiveness of certain anti-dandruff agents with respect to epidermal cells or the ecoflora of the scalp can also induce worsening of the pellicular condition.

Numerous epidermal factors, the expression, biological activity or maturation whereof are altered, reduced or increased, are known to be involved, directly or indirectly, in the renewal, or desquamation, process of the skin, and particularly of the scalp. However, many unknowns still remain as to the intimate mechanism and the factors involved in skin desquamation, and in particular in the onset of dandruff.

Therefore, there is a need to have novel biomarkers suitable for characterizing a pellicular condition of the scalp or predicting the onset of a pellicular condition, particularly helping anticipate recurrences.

This invention aims to respond to this need.

This invention results from the inventors' unexpected discovery that the quantity of the active form of transglutaminase 3 (TGM3) was correlated with the pellicular condition in a scalp. Indeed, they demonstrated a significant increase in the quantity of the active form of TGM3 in dandruff-affected scalp samples compared to normal scalps.

TGM3 catalyzes the formation of isopeptide crosslinks between the glutamine and lysine residues in various proteins, as well as polyamine conjugation with proteins. In hair follicles, it is involved in structural protein crosslinking helping harden the inner sheath of the root.

TGM3 is coded by a 42.8 kb gene containing 13 exons. It is located on chromosome 20: 2 296 001-2 341 079, on the sense strand. TGM3 is produced in the form of a proform having as its amino acid sequence the sequence SEQ ID NO: 3, of 693 amino acids (and having as its nucleotide sequence SEQ ID NO: 4). This enzyme is activated by proteolytic treatment of the proform, generating the active form. The active form thus produced has as its amino acid sequence the sequence SEQ ID NO: 1 of 470 amino acids (and as its nucleotide sequence SEQ ID NO: 2).

The expression of the proform of TGM3 was identified by mass spectrometry as being reduced in dandruff-affected scalps compared to normal scalps (Cavusoglu et al., Archives of Dermatological Research volume 308, pages 631-642(2016). However, the inventors discovered here that the quantity of the active form, detectable by a simple Elisa test, was increased and not reduced in dandruff-affected scalps. However, there is no direct correlation between the quantity of the proform and the quantity of the active form of a protein, these two quantities being dependent on various factors (such as the production rate of the proform, the conversion rate of the proform into the active form, the degradation rate of the proform and the degradation rate of the active form).

This invention thus relates to a method of prognosis and/or diagnosis of a pellicular condition of the scalp, in a subject, said method comprising the following step:
a. measuring the quantity of the active form of transglutaminase 3 (TGM3), in a scalp sample from said subject.

This invention also relates to a method of cosmetic prevention and/or treatment for a scalp having a pellicular condition in a subject, said method comprising the following steps:
a. measuring the quantity of the active form of TGM3, in a scalp sample from said subject;
b. inferring from step a) whether said subject's scalp has a pellicular condition or whether said subject's scalp is likely to develop a pellicular condition;
c. if the scalp is identified as having a pellicular condition or as being likely to develop a pellicular condition in step b), treating said scalp with a cosmetic composition for the pellicular condition.

This invention also relates to a method for identifying a compound for preventing and/or reducing and/or eliminating the pellicular condition of a scalp, said method comprising the following steps:
a. measuring the quantity of the active form of TGM3, in a scalp sample exposed to said candidate compound;
b. comparing said quantity measured in step a) to the quantity of the active form of TGM3, in a scalp sample not exposed to said compound;
c. identifying said candidate compound as a compound for preventing and/or reducing and/or eliminating the pellicular condition of a scalp when a reduction in the quantity of the active form of TGM3 in the scalp sample that was exposed to said candidate compound is detected, with respect to the quantity of the active form of TGM3 in the scalp sample that was not exposed to the candidate compound.

The present invention also relates to a kit comprising:
a. a means for measuring in a scalp sample the quantity of the active form of TGM3, and
b. an instruction leaflet.

This invention also relates to the use of a kit according to the invention for identifying a compound for preventing and/or reducing and/or eliminating the pellicular condition of a scalp.

### DETAILED DESCRIPTION OF THE INVENTION

### Active Form of TGM3

“Active form of active TGM3” means here TGM3 protein after proteolytic cleavage. Preferably, the active form of TGM3 has at least 80%, at least 90% or at least 95% identity with the sequence SEQ ID NO: 1. Preferably, the active form of TGM3 has as its amino acid sequence the sequence SEQ ID NO: 1. It is detectable particularly via specific antibodies of the active form.

“Specific antibody of the active form of TGM3” means an antibody or an antibody fragment which binds with the active form of TGM3.

Antibody fragments include Fab fragment, single chain Fv (scFv), domain antibody, minibody, diabody and triabody. Reference to antibody indicates a full-length antibody; while reference to a fragment thereof indicates a construct comprising at least one of the variable regions as provided in the reference antibody.

In an embodiment, said specific antibody of the active form of TGM3 is able to bind to an active form of a recombinant TGM3 protein produced by eukaryotic cells, and for example produced by animal cells or plant cells. In a preferred embodiment, said specific antibody is able to bind to an active recombinant form of TGM3 produced by insect cells, such as the product T024 from Zedira.

Preferably, said specific antibody of the active form of TGM3 is generated using HuCAL technology.

In brief, in the HuCAL technology, a bank containing antibodies which are presented on phage particles, is screened with the active form of human recombinant TGM3 protein. Then several amplification cycles are performed in E. coli using an auxiliary phase to enrich the antibody phage selected. The DNA of the selected antibody phages is collected and subcloned in a Fab—Fab-A_FSx2 format—bivalent Fab fusion antibody—bacterial alkaline phosphatase expression vector, tracked by markers. After transformation and expression in E. coli, the DNA of the positive clones is selected and sequenced to identify unique antibody clones. Each unique clone can then be expressed, for example in E. coli, and purified. The affinity and the specificity of each antibody can be evaluated by direct ELISA, using a recombinant active form of TGM3 and using non-related antigens and markers.

Thus, in a particular embodiment said specific antibody of fragment thereof of the active form of TGM3 is generated by a process comprising:
a. screening of a bank containing antibodies which are presented on phage particles with the active form of human recombinant TGM3 protein,
b. performing at least two amplification cycles in E. coli using an auxiliary phase to enrich the antibody phage selected,
c. collection of the DNA of the selected antibody phages and subcloning in a Fab expression vector, tracked by markers or tags,
d. transforming E. coli with the Fab expression vector and expressing the Fab,
e. selection of positives clones and eventually sequencing to identify unique antibody clones,
f. Expressing selected Fab, for example in E. coli and purification.

### Method of Prognosis and/or Diagnosis

This invention thus relates to a method of prognosis and/or diagnosis of a pellicular condition of the scalp.

“Pellicular condition of the scalp” denotes a scalp displaying excessive dryness or excessive sebum secretion, characterized by the presence of dry or greasy dandruff, and possibly pruritus. Dry pellicular conditions are sometimes associated with excessively rapid renewal of the stratum corneum. Dry dandruff flakes are generally small in size, white or gray, and are dispersed on the scalp and clothing generating an unsightly visual effect and are sometimes accompanied by itching. Greasy pellicular conditions are frequently characterized by large, greasy and yellow dander flakes which accumulate to form clumps. They are sometimes accompanied by itching, and burning sensations on the areas affected. Greasy pellicular conditions of the scalp occur, and are amplified upon excessive sebum secretion at the epidermis of the scalp.

The method of prognosis and/or diagnosis of a pellicular condition of the scalp, according to the invention comprises a step a) of measuring the quantity of the active form of transglutaminase 3 (TGM3).

The quantity of the active form of transglutaminase 3 (TGM3) can be measured with any suitable technique. In particular, the quantity can be measured by mass spectrometry or by an antibody based assays, such as ELISA test. Preferably, it is measured using a ligand specific for the active form of TGM3, and in particular a specific antibody against the active form of TGM3, or a fragment of said antibody. In a preferred embodiment of the invention, the quantity of the active form of transglutaminase 3 (TGM3) is measured by an ELISA test.

The subject's scalp sample used in the method of prognosis and/or diagnosis according to the invention, is a sample taken, preferably non-invasively, from the subject's scalp, in particular from the subject's temples. Preferably, the scalp sample comes from the stratum corneum, in particular from the surface of the stratum corneum. Preferably, the scalp sample is taken from an area of the scalp without lesion.

The stratum corneum is the outermost layer of the epidermis, and comprises the skin surface. It is essentially made up of dead cells.

According to an embodiment, the method of prognosis and/or diagnosis according to the invention comprises a step of collecting the scalp sample from the subject, in particular from the surface of the subject's scalp. This step is preferably performed non-invasively, and in particular does not require local anesthesia. According to a preferred embodiment, the step of collecting the sample is performed by rubbing the scalp surface, or using an adhesive surface, for example a D-squame® disc. In the case of the adhesive surface, it is applied onto the scalp and then removed in order to collect a sample of the surface of the scalp.

In a specific embodiment, the scalp sample is thus collected by rubbing the surface of said scalp for example using a cotton bud or a sterile swab, or using an adhesive surface, for example a D-squame® disc. Preferably, the scalp sample is collected using sterile swabs.

Soluble protein extract from the scalp sample can be obtained by solubilization in a buffer. In a preferred embodiment, soluble protein extract from the scalp sample are obtained by solubilization in a buffer comprising Tris, and more particularly a buffer at pH 8 comprising from 10 to 100 mM Tris, and from 100 to 200 mM NaCl, preferably comprising 50 mM Tris, and 150 mM NaCl.

Said soluble protein extract can then be used to measure the quantity of the active form of transglutaminase 3 (TGM3) for example by mass spectrometry or by an antibody based assays, such as an ELISA test.

“Subject” here means a human being, preferably aged 20 to 70 years. Preferably, the subject does not suffer from diseases or conditions affecting the scalp.

In a specific embodiment, the method of prognosis and/or diagnosis according to the invention further comprises the steps of:
(b) comparing the quantity of the active form of TGM3 measured in step (a) with a control, and
(c) based on the comparison with step (b), determining whether said subject's scalp is a pellicular scalp or a scalp likely to develop a pellicular condition.

“Likely to develop a pellicular condition of the scalp” means a scalp that had at least once previously a pellicular condition of the scalp and/or a scalp not yet having dry or greasy dandruff but showing early signs of the onset of dandruff such as itching.

In a specific embodiment, said subject's scalp is diagnosed as having a pellicular condition or as being likely to develop a pellicular condition when the quantity of the active form of TGM3 measured in step (a) in the subject's scalp sample is greater, in particular significantly greater, than a control.

In a particular embodiment, the control is a reference value.

In a specific embodiment, the reference value is determined by the mean value of the quantity of the active form of TGM3 measured in a defined population, for example a population in a defined age-group, and/or having a defined scalp type.

In a specific embodiment, the reference value is between 9 ng/ml and 12 ng/ml of TGM3, such as 10.44 ng/ml of TGM3,

In a specific embodiment, the control is the mean value of the quantity of the active form of TGM3 measured in a population of subjects, in particular subjects as defined hereinbelow, having a normal scalp.

“Normal scalp” means here a scalp which has no signs of a pellicular scalp such as the presence of dry or greasy dandruff or pruritus.

In a specific embodiment, the reference value is determined by an ROC study.

“Significantly greater” and “significantly less” according to the invention means a statistically significant variation of the quantity of the active form of TGM3.

Cosmetic Treatment Method

This invention relates to a method of cosmetic prevention and/or treatment for a scalp having a pellicular condition in a subject, said method comprising the following steps:
a. Measuring the quantity of the active form of TGM3, in a scalp sample from said subject;
b. inferring from step a) whether said subject's scalp has a pellicular condition or whether said subject's scalp is likely to develop a pellicular condition;
c. if the scalp is identified as having a pellicular condition or as being likely to develop a pellicular condition in step b), treating said scalp with a cosmetic treatment for the pellicular condition.

The quantity of the active form of TGM3 is preferably measured as described in the “Method of prognosis and/or diagnosis” section above.

The scalp sample and the soluble protein extract from the scalp sample from said subject and the subject can be as described in the “Method of prognosis and/or diagnosis” section above.

“Cosmetic treatment for the pellicular condition” means here any type of cosmetic treatment (topical or oral for example) that is supposed to prevent, reduce or eliminate the pellicular condition.

Said cosmetic treatment can be applied by any suitable administration route, in particular topically or orally.

Said cosmetic treatment can be a cosmetic composition for preventing, reducing and/or eliminating the pellicular condition, preferably for topical application. It can be presented for example in the form of shampoo or conditioner.

Cosmetic compositions for preventing and/or reducing and/or eliminating the pellicular condition can comprise an anti-dandruff agent chosen from ellagic acid, zinc pyrithione, piroctone olamine, selenium disulfide, and mixtures thereof.

In a specific embodiment, said subject's scalp is diagnosed as having a pellicular condition or as being likely to develop a pellicular condition in step b) when the quantity of the active form of TGM3 measured in step (a) in the subject's scalp sample is greater, in particular significantly greater, than a control, as defined above.

Method for Identifying a Compound for Reducing and/or Eliminating the Pellicular Condition of a Scalp

This invention also relates to a method for identifying a compound for preventing and/or reducing and/or eliminating the pellicular condition of a scalp, said method comprising the following steps:
a. measuring the quantity of the active form of TGM3, in a scalp sample exposed to said compound;
b. comparing said quantity measured in step a) to the quantity of the active form of TGM3, in a scalp sample not exposed to said compound;
c. identifying said compound as a compound for preventing and/or reducing and/or eliminating the pellicular condition of a scalp when a reduction in the quantity of the active form of TGM3 in the scalp sample that was exposed to said candidate compound is detected, with respect to the quantity of the active form of TGM3 in the scalp sample that was not exposed to the candidate compound.

In a specific embodiment, the compound is identified as a compound for preventing and/or reducing and/or eliminating the pellicular condition of a scalp when a significant reduction in the quantity of the active form of TGM3 in the scalp sample that was exposed to said candidate compound is detected, with respect to the quantity of the active form of TGM3 in the scalp sample that was not exposed to the candidate compound.

The identification method according to the invention is preferably implemented ex vivo.

According to an embodiment, the scalp sample used in the identification method according to the invention, is a sample taken, invasively or non-invasively, from the scalp of a subject having a pellicular condition.

According to an embodiment, the scalp sample used in the identification method according to the invention, consists of a cell culture obtained from a skin having body hair or a scalp, cells obtained from a skin having body hair or a scalp, a reconstructed complete scalp, or a human scalp explant.

According to an embodiment, the scalp sample used in the identification method according to the invention is a scalp sample obtained by rubbing the surface of the scalp, for example using a cotton bud or a sterile swab, or using an adhesive surface, for example a D-squame® disc. Preferably, the scalp sample is collected using sterile swabs.

Soluble protein extract from the scalp sample can be obtained by solubilization in a buffer. In a preferred embodiment, soluble protein extract from the scalp sample are obtained by solubilization in a buffer comprising Tris, and more particularly a buffer at pH 8 comprising from 10 to 100 mM Tris, and from 100 to 200 mM NaCl, preferably comprising 50 mM Tris, and 150 mM NaCl.

Said soluble protein extract can then be used to measure the quantity of the active form of transglutaminase 3 (TGM3) for example by mass spectrometry or by an antibody based assay, such as an ELISA test.

Preferably, the scalp sample comes from the stratum corneum, in particular from the surface of the stratum corneum.

The quantity of the active form of TGM3 is preferably measured as described in the “Method of prognosis and/or diagnosis” section above.

“Significant reduction” according to the invention means a statistically significant reduction of the quantity of the active form of TGM3.

In an alternative embodiment, the invention relates to a method for evaluating the effectiveness of a cosmetic treatment for the pellicular condition in a subject, said method comprising, after a step of applying said cosmetic treatment, a step (a) of measuring the quantity of the active form of transglutaminase 3 (TGM3), in a scalp sample from said subject.

Preferably, this evaluation method further comprises the steps of:
b. comparing said quantity measured in step a) with a control;
c. determining whether said cosmetic treatment for the pellicular condition is effective in said subject.

The method for evaluating the efficacy is preferably implemented in vitro or ex vivo.

The subject is preferably a human having a dandruff-affected scalp. The subject is preferably aged 20 to 70 years. Preferably, the subject does not suffer from diseases or conditions affecting the scalp.

The scalp sample and the soluble protein extract from the scalp sample from said subject can be as described in the “Method of prognosis and/or diagnosis” section above.

The quantity of the active form of transglutaminase is preferably measured as described in the “Method of prognosis and/or diagnosis” section above.

In a specific embodiment, the control is the quantity of the active form of transglutaminase 3 (TGM3) in a scalp sample from said subject before application of said cosmetic treatment.

Preferably, said cosmetic treatment for the pellicular condition is evaluated as being effective in said subject if the quantity of the active form of transglutaminase 3 measured in step (a) in the subject's scalp sample is less, in particular significantly less, than the control.

“Significantly less than” according to the invention means a statistically significant reduction of the quantity of the active form of TGM3.

Kit

The invention relates to a kit comprising:
a. a means for measuring in a scalp sample the quantity of the active form of TGM3, and
b. an instruction leaflet.

“Means for measuring in a scalp sample the quantity of the active form of TGM3” denotes a means for detecting or quantifying the active form of TGM3. In a specific embodiment of the invention, this means comprises a ligand specific for the active form of TGM3. In a specific embodiment of the invention, this means comprises a pair of capture and detection antibodies specific for the active form of TGM3.

In a specific embodiment of the invention, this means is a microfluidic cartridge comprising at least one antibody specific for the active form of TGM3. In another embodiment of the invention, this means is an immunochromatographic test comprising at least one antibody specific for the active form of TGM3.

According to an embodiment, the kit according to the invention further comprises a standard range of the active form of TGM3.

According to an embodiment, the kit according to the invention further comprises at least one sample with a known concentration of the active form of TGM3. This sample can be used as a control.

According to an embodiment, the kit according to the invention further comprises a solubilization buffer. Preferably, said buffer comprises Tris. In a particular embodiment, said buffer comprises from 10 to 100 mM Tris, and from 100 to 200 mM NaCl, and more preferably comprises 50 mM Tris, and 150 mM NaCl and is at pH 8.

In a specific embodiment, the kit further comprises at least one means for collecting a sample from the scalp. For example, the kit further comprises a sterile swab, or a cotton bud, or an adhesive surface, for example a D-squame® disc.

The invention also relates to the use of a kit according to the invention for identifying a compound for preventing and/or reducing and/or eliminating the pellicular condition of a scalp.

In particular, said use comprises the measurement of the active form of TGM3 in a scalp sample exposed to said candidate compound.

The invention also concerns the use of kit according to the invention, wherein the kit comprise a sample with a known concentration of the active form of TGM3, preferably corresponding to a reference value, and wherein a candidate compound is identified as a compound for preventing and/or reducing and/or eliminating the pellicular condition of a scalp when the amount of the active form of TGM3 measured in the scalp sample exposed to said candidate compound is inferior to the amount of the active form of TGM3 in the sample with a known concentration of the active form of TGM3.

This invention shall be described in more detail by the figures and the examples hereinbelow.

### FIGURES

FIG. 1 shows the linear regression based on the logarithm of the mean TGM3 measurement with respect to the overall score of the pellicular condition. The logarithm of the mean TGM3 measurements is on the y-axis and the dandruff score is on the x-axis.

FIG. 2 shows the ROC curve of the use of the logarithm of the quantity of TGM3 to detect pellicular conditions of the scalp.

### EXAMPLES

### Example 1: Development of Recombinant Human Antibodies Targeted Against TGM3 and Selection of a Pair of Antibodies

Highly specific recombinant human antibodies against TGM3 were generated using Bio-Rad AbD Serotec HuCAL® technology. In brief, a HuCAL® bank containing 4.5×10¹⁰ antibodies, which are presented on phage particles, was screened with 200 μg of the active form of human recombinant TGM3 protein. Three amplification cycles were performed in E. coli using an auxiliary phase to enrich the antibody phage selected. The DNA of the selected antibody phages was collected and subcloned in a Fab—Fab-A_FSx2 format—bivalent Fab fusion antibody—bacterial alkaline phosphatase expression vector, tracked by FLAG® and a His6 marker. After transformation and expression in E. coli, the DNA of the positive clones was selected and sequenced to identify unique antibody clones. Each unique clone was expressed in E. coli and purified by affinity chromatography. The affinity and the specificity of each antibody was evaluated by direct ELISA, using a recombinant TGM3 and using non-related antigens and markers.

### Example 2: Sandwich Test Based on TGM3 Antibody

Microfluidic Cartridges

Personalized TGM3 microfluidic cartridges were used to measure the quantity of TGM3 in soluble protein extracts from stratum corneum samples collected using sterile swabs from the scalp of individuals with and without dandruff. The recombinant capture antibody was fixed inside microfluidic grooves (NanoEnTek as described in the published patent FR3023486). A standard curve, from 1 to 170 ng/ml, was plotted using a recombinant TGM3 (active form). The proteins from the sample on the sterile swab were solubilized in a buffer (50 mM Tris, 150 mM NaCl, pH 8.0), and 35 μl was injected inside the microfluidic cartridges. Detection was performed using fluorescent beads coated with an anti-TGM3 detection antibody.

### Example 3: Clinical Study

Study Design

This is a single-center, randomized, double-blind comparative study, with 3 parallel groups and under medical supervision. A period of 2 treatment-free weeks is followed by 4 weeks of treatments. The treatments consist of the use of a shampoo. Three shampoos are tested and each condition is monitored on a sample of 20 subjects.

The subjects selected for the study are healthy male or female volunteers, aged from 18 to 60 years inclusive, whose hair length is greater than 2 cm and preferably shorter than shoulder length; having a moderate to severe pellicular condition of the scalp; usually using shampoo 3 times/week and seeking to shampoo three times per week during the study;

Evaluation Criteria

The primary criterion is the overall score of the pellicular condition of the scalp: evaluation of adherent and non-adherent dandruff according to an ordinal scale ranging from 0 to 5 (total dandruff score ranging from 0 to 10), at baseline and after 4 weeks of treatment.

Analysis of Results

The TGM3 protein concentration was converted into a logarithmic scale.

The volunteers were classified as dandruff ‘+’ or ‘−’ according to their overall score of the pellicular condition (adherent+non-adherent dandruff) of the entire scalp area by following these rules:

Dandruff ‘+’=(overall score of pellicular condition)>2.5

Dandruff ‘−’=(overall score of pellicular condition)<1

Linear Regression

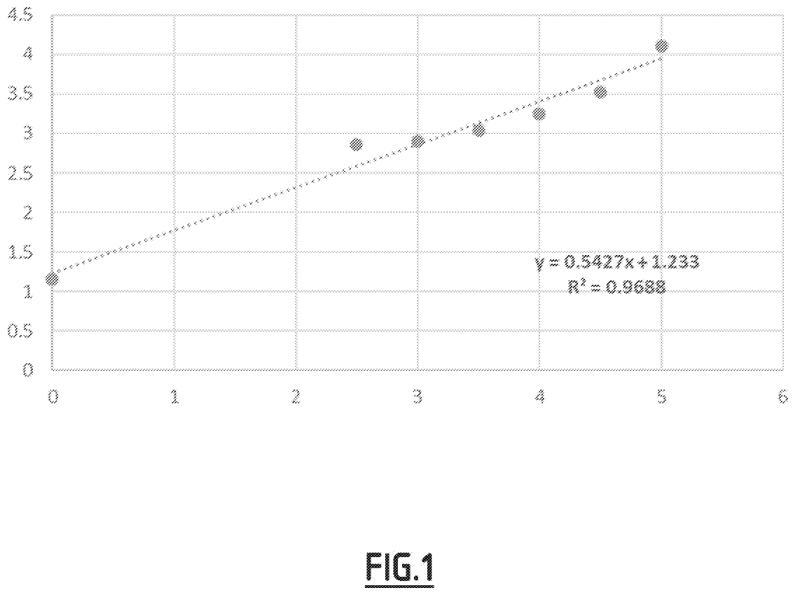

FIG. 1 represents the linear regression based on the mean TGM3 measurement with respect to the overall score of the pellicular condition.

The analysis suggests a good correlation between the TGM3 concentration and the overall score of the pellicular condition with a determination coefficient R² of 0.968.

ROC Curve

The receiver operating characteristic, or ROC curve, is a graphical plot that illustrates the diagnostic ability of a binary classifier system as its discrimination threshold is varied. The rate of true positives is also known as the sensitivity. The rate of false positives is also known as the probability of false alarm and can be calculated as (1 −specificity).

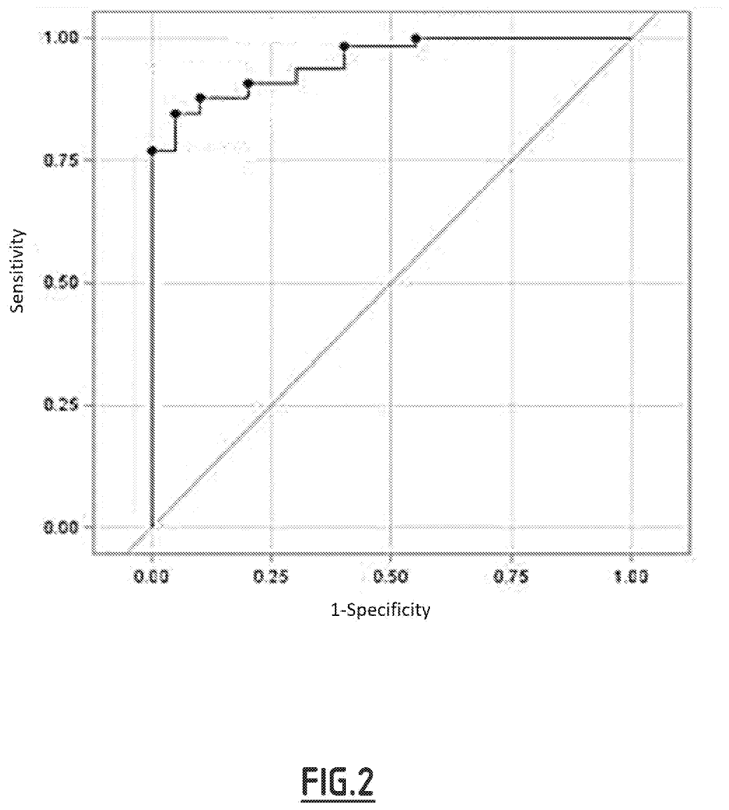

FIG. 2 represents the ROC curve of the use of the logarithm of the quantity of TGM3 to detect pellicular conditions.

This analysis makes it possible to determine the threshold that differentiates the two groups (with and without dandruff) the most and the sensitivity and specificity of the biomarker.

Based on the best sensitivity and specificity of the ROC curve, the threshold for separating the two groups is here 10.44 ng/ml of TGM3 (i.e. 2.346 on a logarithmic scale, corresponding to the third point from the left in FIG. 2).

1. A method of prognosis and/or diagnosis of a pellicular condition of the scalp, in a subject, said method comprising the following step:
a. measuring the quantity of the active form of transglutaminase 3 (TGM3), in a scalp sample from said subject.

2. A method of cosmetic prevention and/or treatment of a scalp having a pellicular condition in a subject, said method comprising the following steps:
a. measuring the quantity of the active form of TGM3, in a scalp sample from said subject;
b. inferring from step a) whether said subject's scalp has a pellicular condition or whether said subject's scalp is likely to develop a pellicular condition;
c. if the scalp is identified as having a pellicular condition or as being likely to develop a pellicular condition in step b), treating said scalp with a cosmetic composition for the pellicular condition.

3. A method for identifying a compound for preventing and/or reducing and/or eliminating the pellicular condition of a scalp, said method comprising the following steps:
a. measuring the quantity of the active form of TGM3, in a scalp sample exposed to said candidate compound;
b. comparing said quantity measured in step a) to the quantity of the active form of TGM3, in a scalp sample not exposed to said compound;
c. identifying said candidate compound as a compound for preventing and/or reducing and/or eliminating the pellicular condition of a scalp when a reduction in the quantity of the active form of TGM3 in the scalp sample that was exposed to said candidate compound is detected, with respect to the quantity of the active form of TGM3 in the scalp sample that was not exposed to the candidate compound.

4. The method according to claim 3, wherein said scalp sample used in the identification method according to the invention, consists of a cell culture obtained from a skin having body hair or a scalp, cells obtained from a skin having body hair or a scalp, a reconstructed complete scalp, or a human scalp explant.

5. The method according to claim 1, wherein the scalp sample is taken by rubbing the scalp surface or using an adhesive surface.

6. The method according to claim 1, wherein the quantity of the active form of TGM3 is measured using a ligand specific for the active form of TGM3.

7. A kit comprising:
a. a means for measuring in a scalp sample the quantity of the active form of TGM3, and
b. an instruction leaflet.

8. The kit according to claim 7, wherein the means for measuring, in a scalp sample, the quantity of the active form of TGM3, comprises a ligand specific for the active form of TGM3.

9. The kit according to claim 7, wherein the means for measuring in a scalp sample comprises an immunochromatographic test.

10. A method for identifying a compound for preventing and/or reducing and/or eliminating the pellicular condition of a scalp which comprises using the kit according to claim 7 to measure the quantity of the active form of TGM3 in a scalp sample exposed to a candidate compound.

11. The method according to claim 2, wherein the scalp sample is taken by rubbing the scalp surface or using an adhesive surface.

12. The method according to claim 3, wherein the scalp sample is taken by rubbing the scalp surface or using an adhesive surface.

13. The method according to claim 4, wherein the scalp sample is taken by rubbing the scalp surface or using an adhesive surface.

14. The method according to claim 2, wherein the quantity of the active form of TGM3 is measured using a ligand specific for the active form of TGM3.

15. The method according to claim 3, wherein the quantity of the active form of TGM3 is measured using a ligand specific for the active form of TGM3.

16. The method according to claim 4, wherein the quantity of the active form of TGM3 is measured using a ligand specific for the active form of TGM3.

17. The method according to claim 5, wherein the quantity of the active form of TGM3 is measured using a ligand specific for the active form of TGM3.

18. The kit according to claim 8, wherein the means for measuring in a scalp sample comprises an immunochromatographic test.

19. A method for identifying a compound for preventing and/or reducing and/or eliminating the pellicular condition of a scalp which comprises using the kit according to claim 8 to measure the quantity of the active form of TGM3 in a scalp sample exposed to a candidate compound.

20. A method for identifying a compound for preventing and/or reducing and/or eliminating the pellicular condition of a scalp which comprises using the kit according to claim 9 to measure the quantity of the active form of TGM3 in a scalp sample exposed to a candidate compound.
